(19) United States
(12) Patent Application Publication
(10) Pub. No.: US 2006/0155339 A1
(43) Pub. Date: Jul. 13, 2006

(54) Writer-mode ecg display

(76) Inventors: Kai Hensen, Juelich (DE); Michael Koinzer, Bergholz-Rehbruecke (DE); Stefan Letzner, Blankenfelde (DE)

Correspondence Address: HAHN LOESER & PARKS, LLP, One GOJO Plaza, Suite 300, AKRON, OH, 44311-1076, US

(21) Appl. No.: 10/526,136

(22) PCT Filed: Aug. 28, 2003
(86) PCT No.: PCT/EP03/09517

§ 371 Date: Jan. 31, 2006

(30) Foreign Application Priority Data
Aug. 29, 2002 (DE) 202 13 612.4

(51) Int. Cl. A61N 1/372

(52) U.S. Cl. 607/32

(57) ABSTRACT

An external programming device for an implant comprises a receiving unit for receiving data from the implant and a display with an actuating unit which are adapted to represent signals forming the basis of the received data. A switching unit connected to the actuating unit of the display is provided to switch over the representation of continuous signals over time, between a first and at least one second representation mode. Representation of the signals is effected in one mode continuously in that current display values are always represented at the same horizontal display position and all preceding signal values are represented displaced horizontally towards the left or the right on the display. Representation of the signals is effected in the another continuously in that respective current signal values are represented at a new display position of the display adjoining preceding signal values, while preceding signal values maintain their respective display position.

### BACKGROUND OF THE INVENTION

The invention concerns an external programming device for an implant such as a cardiac pacemaker, defibrillator or the like, comprising a receiving unit for receiving data from the implant, which represent time-variable signals which are intracardially recorded or generated in the implant, such as an intracardial electrocardiogram (ECG), and a display with actuating unit which are adapted to represent signals forming the basis of the received data, such as, for example, the intracardial ECG.

Such programming devices are basically known but they frequently leave something to be desired in regard to the representation of intracardial electrocardiograms for the physician conducting the treatment.

### BRIEF SUMMARY OF THE INVENTION

The object of the present invention is to provide a device which is more suitable in that respect.

In accordance with the invention, in a programming device of the kind set forth in the opening part of this specification, there is provided a switching unit which is connected to the actuating unit of the display and which is adapted to switch over the representation of continuous signals over time such as the intracardial ECG between a first and at least one second representation mode, wherein representation of the continuous signals over time is effected in the first mode continuously in that current signal values are respectively represented at a new display position of the display adjoining preceding signal values, while preceding signal values retain their respective display position, and wherein representation of the continuous signals over time is effected in the second mode continuously in that current display values are respectively represented at always the same horizontal display position and representation of all preceding signal values, with representation of a respective new current signal value, are represented on the display displaced horizontally towards the left or the right.

Preferably, representation of the display values is effected in the first mode continuously from left to right in such a way that signal values which have already been represented retain their representation location and the representation is prolonged in each case with each arriving signal value, starting from a left-hand representation edge, until the representation of the signal values has reached a right-hand representation edge.

In that respect, a particularly preferred programming device is one in which representation is extinguished when the representation edge is reached and is begun afresh with a respective current signal value at the left-hand representation edge.

A preferred programming device is one in which the representation of the display values is effected in the second mode continuously, in that respectively current signal values are represented at a right-hand representation edge and preceding signal values are moved at the same time towards the left by a display position, but are not represented beyond a left-hand representation edge.

A programming device which is particularly preferred is one in which the switching unit is connected to a switching element in such a way that switching from the first mode to the second mode and vice-versa is effected by touching or pressing the switching element.

In an alternative configuration, the switching element can be a press switch arranged beside the display.

Alternatively, the switching element is formed by a defined region (switching surface) of the display, wherein the display at least in that region is touch-sensitive or pressure-sensitive.

### BRIEF DESCRIPTION OF THE SEVERAL VIEWS OF THE DRAWINGS

In the drawings:

FIG. 1 shows a front view of the programming device with the base device and the hand device removed,

FIG. 2 shows a rear view of the programming device with the hand device coupled to the base device,

FIG. 3 shows a diagrammatic block representation of the functional components of the hand device and the base device,

FIG. 4 shows a diagrammatic representation of a first display mode for the representation of intracardial cardiograms on the display of the hand device, and

FIG. 5 shows a representation of a second display mode for the representation of intracardial cardiograms on the display of the hand device.

### DETAILED DESCRIPTION OF THE INVENTION

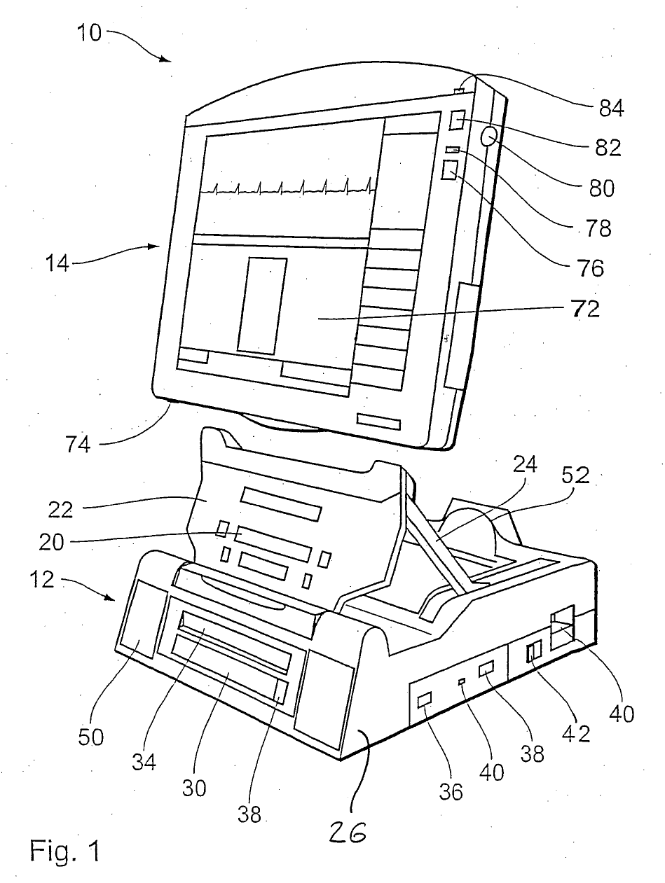

The programming device 10 shown in FIG. 1 is of a two-part modular structure and includes a base device 12 and a hand device 14.

As can be seen from FIG. 1, the base device 12 and the hand device 14 can be separated from each other.

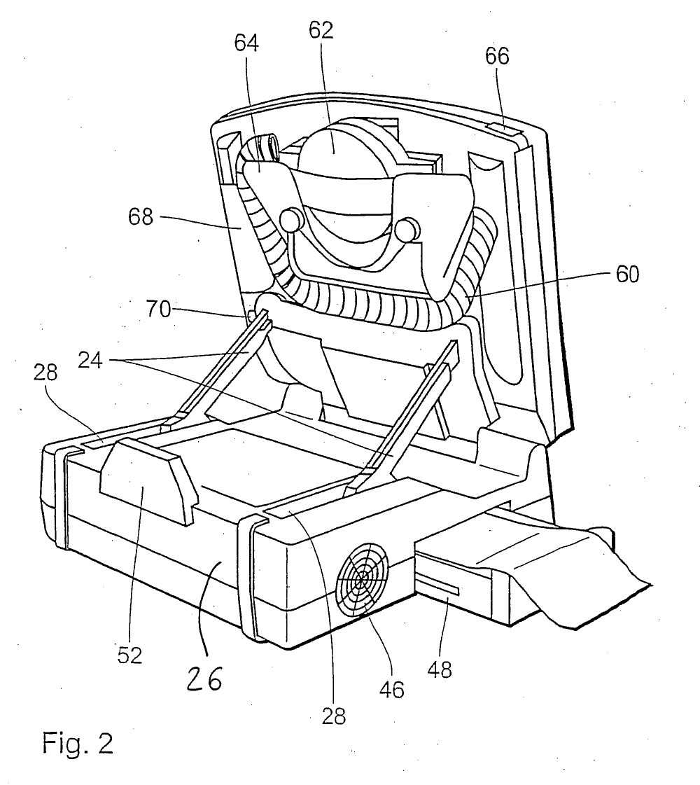

FIG. 2 is a rear view of the base device 12 and the hand device 14 in a condition in which the two devices are coupled together.

For electrically coupling the hand device 14 to the base device 12, the latter has a central plug connection 20. That central plug connection 20 is disposed on the front side of a mounting tilting member 22 which also serves for mechanically mounting the hand device 14. The mounting tilting member 22 is pivotably mounted to a front side of the base device 12 and is supported by two supports 24, at its side remote from its pivotal mounting to the base device 12. Those two supports 24 are pivotably attached to the mounting tilting member 22. At their respective other ends, the supports 24 are longitudinally displaceably connected to a main body of the base device 12 so that adjusting of the angle between the main body 26 of the base device 12 and the mounting tilting member 22 is possible by longitudinal displacement of the supports 24 with respect to the main body 26 of the base device 12. For that purpose, the main body 26 of the base device 12 has longitudinal guides 28 for the supports 24.

The following further components are disposed in the main body 26 of the base device 12: a carrying handle 30 which is lockable in the main body 26 and which issues somewhat from the main body 26 under a spring force by pressing a button 34. In addition, the main body 26 includes a CD-ROM drive 34 as well as a USB interface 36, a serial interface 38, a mains power connection 40, an on switch 42 which is to be actuated when the battery is discharged, an operating display 44, a fan with fan cover 46 and a printer with an extendable paper supply container 48. The printer is to be operated by way of a printer keyboard 50.

There is also a support leg 52 on which the coupled hand device 14 can rest when the mounting tilting member 22 is laid entirely flat.

As shown in FIG. 1, the hand device 14 may be uncoupled from the base device 12 and used autonomously. For that purpose, the hand device 14 has a battery power supply disposed behind a cover 58. The hand device 14 is connected by way of an electrical feed line 60 to a programming head 62. The programming head 62 is adapted to be placed on the skin of a patient in order to make a telemetric connection which is as short as possible with an implant of the patient. A suitable holder 64 is provided on the rear side of the hand device 14 for receiving the programming head 62. The arrangement also has a connecting socket for the electrical feed line 60, which in FIG. 2 is covered by the programming head 62, in order to be able to separate the programming head 62 together with the feed line 60 from the hand device 14.

Provided on the rear side of the hand device 14 is a counterpart plug connection (not shown in the Figures) which, in the coupled condition of the hand device 14 shown in FIG. 2, engages into the central plug connection 20 in the mounting member 22 of the base device 12. In the coupled condition moreover, the hand device 14 is mechanically locked in a known manner to the mounting tilting member 22. That locking action is to be released by means of a locking button 68.

The hand device 14 also has a fan disposed behind a fan cover 70.

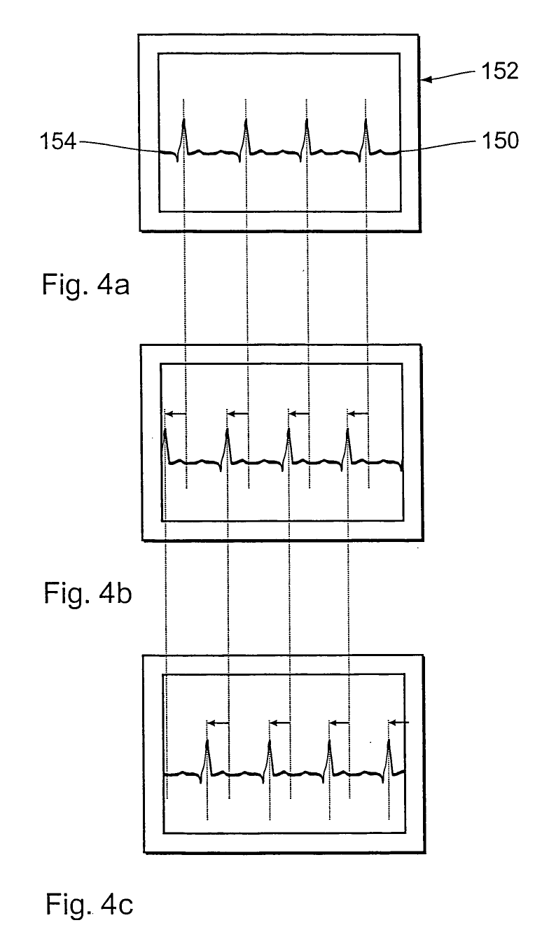

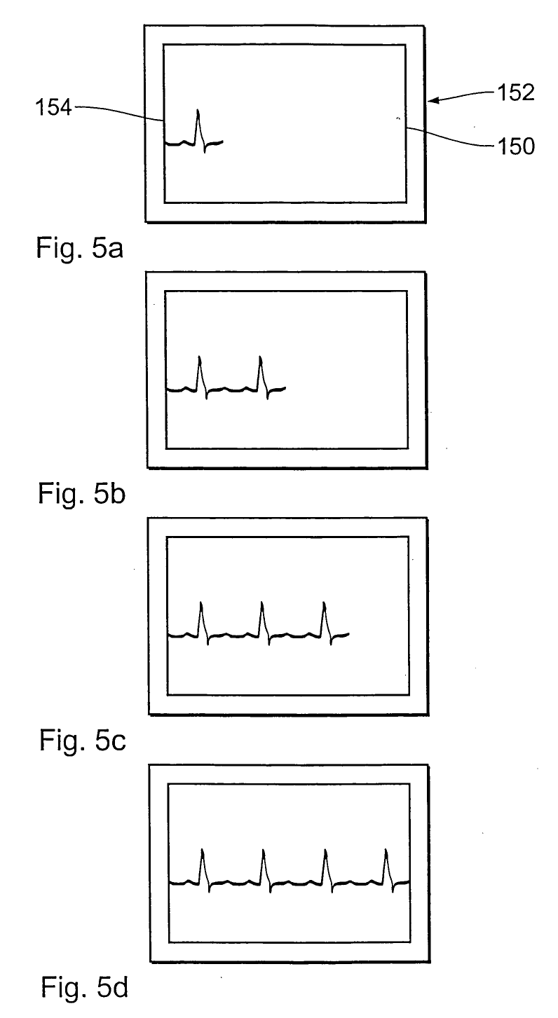

Provided on the front side of the hand device 14 as an essential component is a display 72 in the form of a touch screen. As the screen 72 is touch-sensitive, it can represent in a known manner programmed switching surfaces which upon actuation, lead to corresponding input signals from the hand device 14. Such a programmed switching surface is that for switching over between two representation modes for the representation of intracardial cardiograms which will be described in greater detail hereinafter with reference to FIGS. 4 and 5.

Further components of the hand device 14 are an emergency shock button 74, with which the implant of a patient can be energised from the hand device 14 by way of the programming head 10 to deliver a defibrillation pulse.

There is also provided a charge condition button 76, upon the actuation of which, the charge condition of the battery power supply of the hand device 14 is displayed. That is effected by way of a charge condition display 78.

The hand device 14 further has a socket 80 for connecting a device for recording surface electrocardiograms.

It will be appreciated that the hand device 14 also has an on switch 82 and an operating display 84.

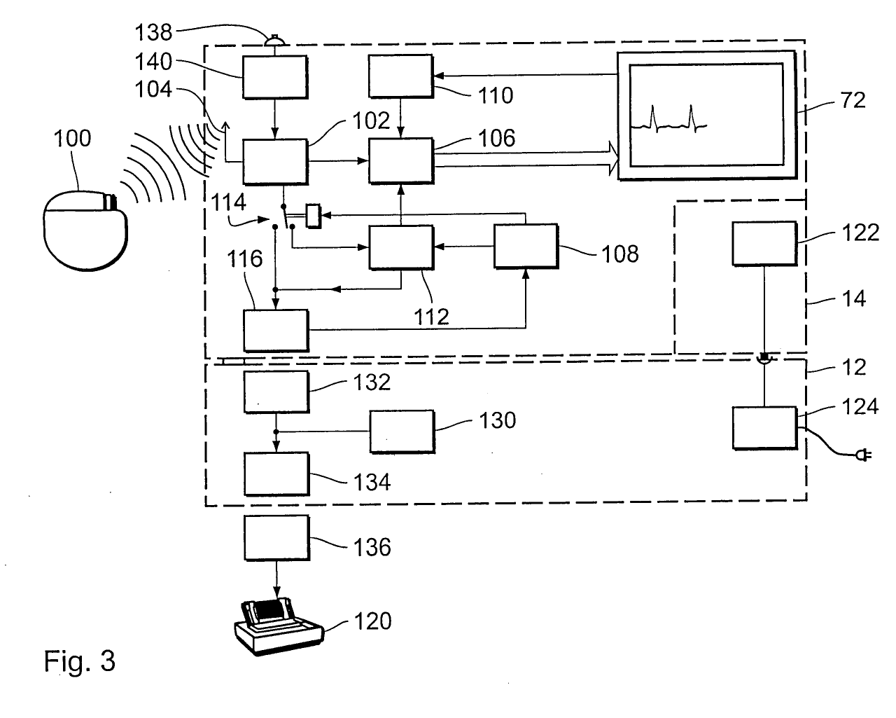

FIG. 3 shows a highly diagrammatic view of some of the internal components of the hand device 14 and the base device 12 as well as an implant 100. The hand device 14 is adapted to make a bidirectional telemetric connection between the implant 100 and the hand device 14. For that purpose the hand device 14 includes a transmitting and receiving unit 102 which, like an antenna 104, are a component part of the programming head 62. Signals on the part of the implant, which are recorded by the transmitting and receiving unit 102, are passed on the one hand to a display control unit 106 which actuates the display screen 72 for the display for example of electrograms intracardially recorded by the implant 100. The display control unit 106 is also connected to a central control unit 108 so that any symbol indicated by the central control unit 108, inter alia switching surfaces or text labels, can be represented on the display screen 72.

As the display screen 72 is in the form of a touch screen, it is connected to a detection unit 110 which, when the display screen 72 is touched, generates a signal corresponding to the location at which it was touched. The evaluation unit 110 is also connected to the control unit 106.

Depending on respective actuation by the central control unit 108, signals are passed from the transmitting and receiving unit 102 directly to the actuating unit 106 or to a data memory 112. In addition the central control unit 102 can provide, by way of a switch 114, that data from the transmitting and receiving unit 102 are applied directly to a central connection plug 116 of the hand device 14. The central connecting plug 116 is connected to the central connection plug 20 of the base device 12, when the hand device 14 is coupled to the base device 12.

For receiving the data from the hand device 14, the base device 12 has a second data interface 132 connected to a printer interface 134. There is also provided a base device data memory 130 with which data can be put into intermediate storage, between the data interface 132 and the printer interface 134. A printer 120 can be connected with its interface 136 to the base device 12, by way of the printer interface 134. In a preferred embodiment which is illustrated in FIGS. 1 and 2 the printer is integrated into the base device 12.

In order to actuate the implant 100 under remote control by way of the hand device 14 for the delivery of a defibrillation shock, the hand device 14 has the shock triggering button 138 (emergency shock button 74 in FIG. 1) which is connected to the transmitting and receiving unit 102 of the hand device 14 by way of a suitable shock actuation unit 104.

The central control unit 108 is also connected to the central connection plug 116 of the hand device 14 in such a way that the central control unit 108 independently detects when the hand device 14 is coupled to the base device 12. In that situation the central control unit 108 automatically produces a data connection between the transmitting and receiving unit 102 and the central connection plug 116 in order to provide a real-time print-out of an intracardial electrocardiogram received by the transmitting and receiving unit 102, on a printer 120 of the base device 12.

When the hand device 14 is coupled, the battery power supply 122 of the hand device 14 is also electrically connected to a mains power supply 124 of the base device 12 in order to charge up the battery of the hand device 14.

The central control unit 108 has a program memory and is so programmed that, upon the display of intracardial electrocardiograms on the display screen 72, beside the represented electrocardiogram, there also appears a switching surface with which it is possible to switch over the display of intracardial electrocardiograms on the display screen 72. If the act of touching the switching surface for switching over the representation mode is detected by way of the evaluation unit 110 and the actuating unit 106 as well as the central control unit 108, the representation mode is switched over from a first to a second mode or vice-versa.

The two representation modes are shown in FIGS. 4 and 5. In the first representation mode shown in FIGS. 4a through 4c, a respectively current signal value of the intracardial electrocardiogram is represented at a right-hand representation edge 150 of a representation window 152 on the display screen 72. All preceding signal values of the ECG are represented further to the left thereof, more specifically in such a way that the oldest represented ECG signal value is at the left-hand representation edge 154. With the representation of each new ECG signal value at the right-hand representation edge 150, all earlier ECG signal values are displaced towards the left by a display screen position. That is symbolically represented in FIGS. 4b and c. That gives the impression that the ECG displayed in the first representation mode in the display screen 72 runs continuously from right to left through the representation window 152 on the display screen 72.

In the second representation mode illustrated in FIGS. 5a through d, the electrocardiogram, starting from the left-hand representation edge 154, is continuously written along as far as the right-hand representation edge 150, insofar as each new ECG signal value is attached to the ECG signal values which have already been represented, in a condition of being displaced towards the right by a horizontal display screen position. When, as shown in FIG. 5d, the representation has reached the right-hand display screen edge 150, the display is extinguished and the procedure of progressively writing the ECG at the left-hand representation edge 154 is begun afresh.

By virtue of the fact that the display of electrocardiograms can be switched over between the two above-described representation modes, it is possible for the first time for the physician in charge of the treatment to freely select the mode of representation which is respectively appropriate for observing the phenomenon in which he is interested.

When the hand device 14 is coupled to the base device 12, the physician also receives a print-out of the respective current ECG in real time. The fact that the hand device 14 can be uncoupled from the base device 12 also affords the physician the possibility of moving autonomously in space with the hand device 14 while in that case nonetheless having all essential functions and modes of representation available to him.

1. An external programming device for an implant comprising:
a receiving unit for receiving data from the implant, which represent time-variable signals which are intracardially recorded or generated in the implant, and
a display with an actuating unit adapted to represent signals forming the basis of the received data, and
a switching unit which is connected to the actuating unit of the display and adapted to switch the representation of continuous signals over time between a first representation mode and at least one second representation mode,
wherein representation of the time-continuous signals over time is effected in the first mode continuously in that current display values are always represented at the same horizontal display position and of all preceding signal values are represented on the display, displaced horizontally towards the left or the right, and
wherein representation of the continuous signals over time is effected in the second mode continuously in that current signal values are respectively represented at a new display position of the display adjoining preceding signal values while preceding signal values maintain their respective display position.

2. A programming device as set forth in claim 1, wherein representation of the display values in the second mode is effected continuously from left to right in that signal values which have already been represented maintain their representation location and the representation is respectively extended with each arriving signal value, starting from a left-hand representation edge until the representation of the signal values has reached a right-hand representation edge.

3. A programming device as set forth in claim 2, wherein the representation is extinguished when the right-hand representation edge is reached and is begun afresh with a respectively current signal value at the left-hand representation edge.

4. A programming device as set forth in claim 1, wherein the representation of the display values in the first mode is effected continuously in such a way that respective current signal values are represented at a right-hand representation edge and preceding signal values are simultaneously displaced towards the left by a display position but are not represented beyond a left-hand representation edge.

5. A programming device as set forth in claim 1, wherein the switching unit is connected to a switching element in such a way that switching from the first to the second mode and vice-versa is effected by touching the switching element or by pressing on the switching element.

6. A programming device as set forth in claim 5, wherein the switching element is a press switch arranged beside the display.

7. A programming device as set forth in claim 5, wherein the switching element is formed by a defined region of the display and the display is touch-sensitive or pressure-sensitive at least in that region.
